(19) United States
(12) Patent Application Publication
(10) Pub. No.: US 2016/0089027 A1
(43) Pub. Date: Mar. 31, 2016

(54) METHOD FOR PHOTOGRAPHICALLY OBSERVING AND/OR DOCUMENTING THE FUNDUS OF AN EYE, AND FUNDUS CAMERA

(71) Applicants: VOIGTMANN GMBH, Nürnberg (DE); TALKINGEYES & MORE GMBH, Erlangen (DE); FRIEDRICH-ALEXANDER-UNIVERSITÄT ERLANGEN-NÜRNBERG, Erlangen (DE); UNIVERSITÄTSKLINIKUM ERLANGEN, Erlangen (DE)

(72) Inventors: Peter VOIGTMANN, Rückersdorf (DE); Bernhard HOHER, Erlangen (DE); Bernhard SCHMAUSS, Theisseil (DE); Georg MICHELSON, Baiersdorf (DE); Thomas KOHLER, Erlangen (DE)

(73) Assignees: VOIGTMANN GMBH, Nurnberg (DE); TALKINGEYES & MORE GMBH, Erlangen (DE); FRIEDRICH-ALEXANDER-UNIVERSITAT ERLANGEN-NURNBERG, Erlangen (DE); UNIVERSITATSKLINIKUM ERLANGEN, Erlangen (DE)

(21) Appl. No.: 14/782,752

(22) PCT Filed: Apr. 8, 2014
(86) PCT No.: PCT/EP2014/057069

§ 371 Date: Oct. 6, 2015

(30) Foreign Application Priority Data
Apr. 8, 2013 (DE) 10 2013 005 869.4

(51) Int. Cl. A61B 3/15; A61B 3/00; A61B 3/12

(52) U.S. Cl. CPC A61B 3/158; A61B 3/12; A61B 3/0025; A61B 3/0091

(57) ABSTRACT

The invention relates to a method for photographically observing and/or documenting the fundus of an eye, comprising: producing a lighting beam path by means of a lighting module for lighting at least one partial area of the fundus by using an at least approximately punctiform or at least areally limited lighting source, producing an image plane in the lighting beam path for the creation of photographic recordings, separating the pupil cross-sectional area into a detectable observation region and an undetectable lighting region, producing an image area that contains the image of the fundus on the image plane, dividing the image area in strips in the lighting beam path into a lighted region and unlighted regions, and imaging the third and fourth Purkinje reflection on the image area, wherein at least one Purkinje reflection, the third and/or fourth Purkinje reflection, is located in the unlighted regions of the image area.

The present invention concerns a method for photographic observation and/or documentation of the fundus of the eye, as well as a fundus camera for producing such photographs, preferably without the use of a mydriatic agent, i.e., a medication widening the pupil. Furthermore, the present invention concerns a patient guidance module for use in a fundus camera.

### TECHNOLOGICAL BACKGROUND

With a fundus camera, also known as a “retina camera” or “ophthalmoscope”, photographic recordings are made of the fundus (back of the eye). Such recordings are used in support of ophthalmologic diagnostics. They serve to display and document pathological changes in the retina. A fundus camera is outfitted with a lighting source, a lighting optics, and a digital camera, making it possible to prepare high-resolution photographs, which can be stored in a digital patient record or printed out and placed in the patient's file.

On account of the lighting, there are artifacts in a fundus camera due to back reflections of the lighting source at various places in the eye. These back reflections are also called Purkinje reflections. The position, configuration, and cause of these Purkinje reflections are known from U.S. Pat. No. 4,729,652, to which reference is made for its entire contents. The first, second and fourth Purkinje reflections lie at the points of their sharp focus in roughly the region of the lens of the eye. The third Purkinje reflection lies at the point of its sharp focus roughly between the lens and the fundus.

The fundus cameras currently on the market can be divided into the following different groups according to the method of suppressing artifacts.

The first group of instruments uses an annular pupil division. While the pupil is illuminated by an outer lighting ring, the detection of the light back scattered by the fundus occurs through a circular middle zone of the pupil of the eye, free of the illumination light. For the separation of the outer lighting ring from the inner detection region of the pupil of the eye, a narrow transition zone is provided between the two regions, in which neither lighting nor detection occurs. This transition zone is useful, since it can provide a complete separation of lighting and detection beams not only in the corneal plane but also in the entire anterior chamber of the eye, i.e., from the front side of the cornea to the back side of the lens of the eye. With such fundus cameras one can record pictures of the fundus which are free of Purkinje reflections on account of the separation of lighting and detection, but the fundus angle which can be achieved is dependent on the diameter of the pupil, due to the annular pupil division. In a non-mydriatic eye, i.e., an eye not previously treated with a pupil widening medication (mydriaticum), the detection field is very limited.

Furthermore, there are methods in which polarized light is used in order to prevent or reduce back reflections, especially those back reflections which arise within the optics of the fundus camera.

Moreover, there are so-called line scanning methods in which a shiny line is produced on the fundus, which is moved (scanned) across the fundus perpendicular to its orientation. During this process, the fundus is mapped on a digital camera, so that a complete fundus picture is exposed.

### CLOSEST PRIOR ART

From WO 2012/059236 A1 there is already known a fundus camera with striplike pupil division and a method for recording of artifact-free, high-resolution fundus images. A striplike pupil division of the eye being examined is produced here in the form of a vertical bar, characterizing the lighting zone, and circular segments on either side representing the detection zones of the pupil. Furthermore, with the aid of a movable slit diaphragm of the lighting source the lighting is limited to a slit form and scanned across the fundus of the eye. The light reflected from the retina impinges as an image of the slit on the respective sectors of a position-resolving detector and can be read off there. Purkinje reflections are also recorded in this method. In order to avoid these reflections in the final recordings, from each bright image recorded there is taken a second picture (dark image), which is subtracted from the bright image in order to eliminate interference reflections.

### PROBLEM SOLVED BY THE PRESENT INVENTION

The problem to be solved by the present invention is to provide a method for the photographic observation, documentation and/or diagnosis of the aforementioned kind, by means of which on the one hand a very large region of the retina can be recorded without administering a mydriaticum and on the other hand the use of a particularly economical variant of a fundus camera is made possible and the least possible amount of light is beamed into the eye of the patient in order to minimize the light burden on the patient.

### SOLUTION OF THE PROBLEM

The above problem is solved in the method of this kind by the features of the method according to claim 1. In regard to the fundus camera, the problem is solved by a fundus camera according to the features of claim 17.

Expedient embodiments of the invention are proposed in the dependent claims.

The method according to the invention per claim 1 enables the recording of striplike regions of the retina, combined with the possibility of a very simple and economical instrument design. The method moreover opens up the possibility of recording a very large region of the retina along the lengthwise axis of the strip. Increased patient comfort is created thanks to the option of not using a mydriaticum.

Because the detectable observation region and the undetectable lighting region can have a semicircular or circular section shape, especially in combination with an approximately punctiform or at least areally bounded lighting source with a high radiant power per surface area, it is possible to project the third and/or fourth Purkinje reflection advantageously into unused regions.

Insofar as the aperture plane A1 is displaced from the pupil back into the eye, preferably into a plane at the front side of the lens of the eye or at least into a plane in the region of the lens of the eye, the front lens surface is crossed by a convergent beam and the rear lens surface by a divergent beam. Thanks to the opposite curvature of the surfaces of the lens of the eye, each time in the reflection of the lighting rays there occurs a parallelization of the reflected beam. Thus, the aperture plane A1 is projected to infinity, so that just like the fundus image it has “infinite” focal position. This results in an at least essentially sharp projection of the aperture plane A1 and thus also the light source on the light-sensitive electronic component of the digital camera. In this way, artifacts such as the third and/or fourth Purkinje reflection are reduced to especially small surface regions in the fundus image and can therefore be accommodated in the marginal regions of the striplike tripartite division of the image area.

The striplike illuminated region of the image area corresponds in the method of the invention to an image angle in the transverse direction to the strip of at most 5° to 30°, preferably 18° to 24°, especially preferably 20° to 22°.

Likewise, the striplike illuminated region of the image area corresponds to an image angle in the lengthwise direction to the strip of 40° to 90°, preferably 64° to 72°, especially preferably 66° to 70°. Consequently, the method makes it possible to image a very large region of the retina.

Advisedly, the lighting beam path and/or the detection beam path is stationary relative to the entire layout of the fundus camera during a recording, i.e., it is not exposed to any variable translatory movement relative to each other and relative to the eye. Thus, costly mechanical components are not needed.

Because the surface of the fundus being examined in the lighting beam path and detection beam path is varied by changing the viewing direction of the eye of the patient relative to the orientation of the lighting beam path and/or detection beam path, one can still in this way project regions of the fundus going beyond the illuminated strip with a stationary lighting beam path and/or detection beam path and preferably assemble them into an overall image. In this way, the region of the fundus being examined can be substantially increased. It is likewise possible to reduce the image angle of the illuminated region of the image area in the transverse direction to the strip, for example, to less than 20°, preferably to less than 18°, especially preferably to less than 16°, in order to further reduce interference influences of the light source.

The “guiding” of the human eye is advisedly done by creating a fixation mark, which can change position and be projected on the fundus and is therefore seen.

Optionally, the fixation mark can be recorded and documented by the camera.

Advisedly, in the method of the invention a fixation mark is created for each eye separately and used for the patient guidance. In contrast with the monocular method, the “instrument accommodation” often observed here, i.e., a disruption of the automatic distance rule in the brain, is avoided. Furthermore, this method allows a case by case variation of the accommodation and/or the convergence. Without the use of moving parts, the method of the invention preferably achieves both a variation of the accommodation, by variable focusing, and the convergence by vertical movement of the fixation mark.

The method makes it possible to cover, i.e., to project an overall image in regard to the transverse direction to the trend of the striplike division with an image angle of at least 45°, preferably at least 60°, especially preferably at least 65° of the fundus.

Because the third as well as the fourth Purkinje reflections R3 and/or R4 are sharply projected in the fundus, these can be used as optical reference positions for determining the relative position of the detection optics in regard to the viewing direction of the eye.

The fundus camera according to the invention advisedly comprises an eyepiece with a field diaphragm, by which the image field can be bounded in a wide angle. It is advantageous to use a commercial eyepiece here, e.g., one from the field of amateur astronomy or microscopy, which brings with it the advantage of an optics optimally adapted to the eye as well as low cost due to mass market availability.

The slanting orientation of the display to the image plane B5 makes it possible to change the focus without the use of moving parts (solid state device), by moving horizontally the fixation mark (pixel) indicated on the display, which changes the distance between the fixation mark on the display and the projection lens of the display. This variable focus ability enables an adapting of the patient's accommodation allowing for any visual defects which are present.

The use of the colors infrared, green and ultraviolet (IRGUV) for lighting the fundus offers the advantage that color images can be obtained even when using a black and white digital camera. Because a black and white digital camera has a greater light sensitivity than a color digital camera, the intensity of the illumination and thus the burden on the patient can be reduced when using a black and white digital camera. To produce color images, black and white pictures are recorded in sequence and each time illuminated with different colors, infrared, green, or ultraviolet. The three images produced in this way are coordinated with the three primary colors of red, green and blue and reconstructed by additive color mixing on the computer. This method produces less stress on the patient due to the reduced light exposure as well as a reduction or avoidance of annoying ghost images. Due to the lower illumination intensity, moreover, a reduction of the influence of the pupil closure reflex is achieved, so that better images can be achieved thanks to a larger pupil. In addition, an infrared preview mode is easily possible for the orientation and centering of the fundus camera.

According to one embodiment of the invention, several gray-scale images are taken in sequence, each time there being active only a single LED or light source.

According to another embodiment of the invention, one or more dark reference images are recorded for each emission wavelength of the light source, replacing the eye with a light absorber.

According to another embodiment of the invention, one or more bright reference images are recorded for each emission wavelength of the light source, replacing the eye with a diffuse reflector which serves as a white reference.

According to another embodiment of the invention, artifacts in the fundus images and image noise is at least partially removed by subtracting each time from the fundus images a dark reference image or the averaging of several dark reference images.

According to another embodiment of the invention, a color matching and a reduction of image noise can be accomplished by a pixel by pixel division of a fundus image by a bright reference image or by the averaging of several bright reference images.

According to another embodiment of the invention, several gray-scale images of the fundus, each recorded at different emission wavelengths of the light source, are combined into a color image or multispectrum image, preferably performing a relative displacement and/or rotation and/or scaling of the gray-scale images in advance, so that in the combined image the represented structures of the fundus are brought into alignment.

According to another embodiment of the invention, several gray-scale images, color images, false color images or multispectrum images are set off against each other so that the resolution is enhanced and/or the noise component is decreased and/or the visible region of the fundus is enlarged.

According to another embodiment of the invention, a video sequence is produced from several fundus images which were produced by one or more of the aforementioned methods. This has the advantage that the method of the invention can also be combined with investigation techniques (such as fluorescence angiography), preferably at the same time.

The also claimed patient guidance module allows a guiding of the patient's eye while at the same time enabling a changing of the accommodation and balancing out of vision defects by focusing without moving parts in the instrument design.

### DESCRIPTION OF THE INVENTION BY SAMPLE EMBODIMENTS

In the figures of the drawing, sample embodiments of the present invention are explained more closely in detail.

There are shown:

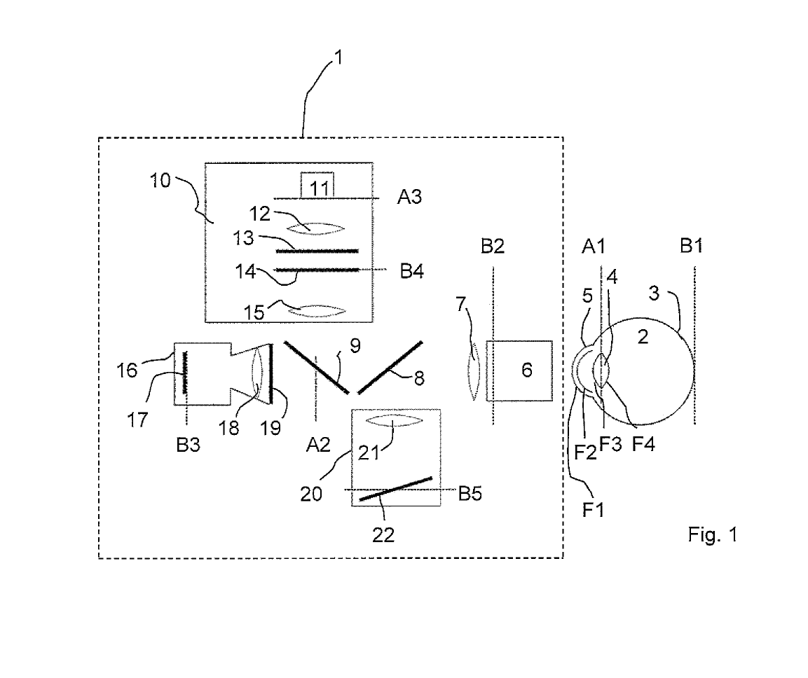

FIG. 1, a representation of a first example of a fundus camera according to the invention;

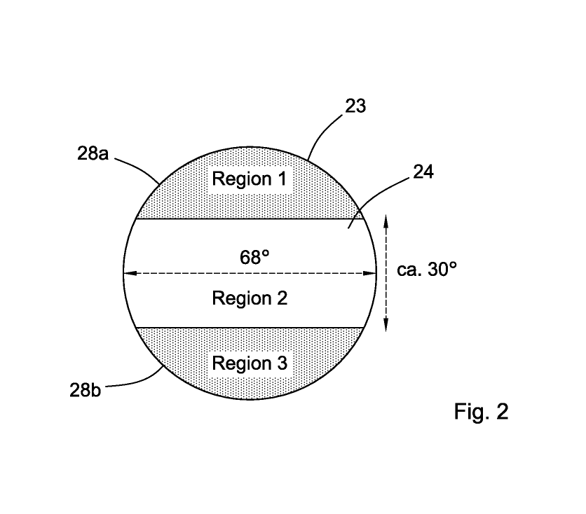

FIG. 2, a representation of the striplike division of the image area into lighted and unlighted regions for the fundus camera of FIG. 1;

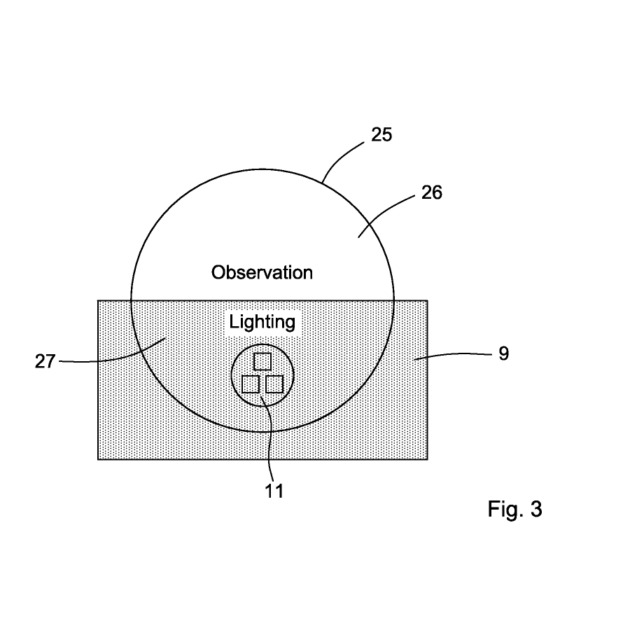

FIG. 3, a division of the aperture (pupil) for the fundus camera of FIG. 1;

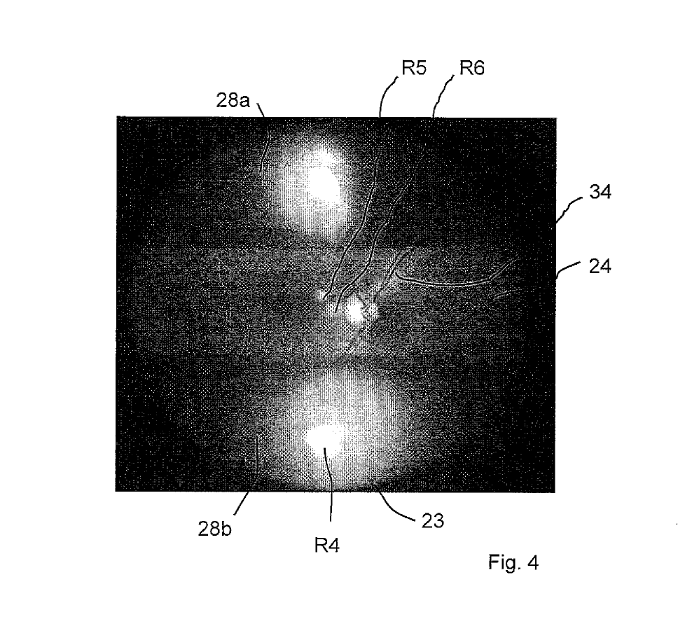

FIG. 4, a photographic recording of the image area per FIG. 2;

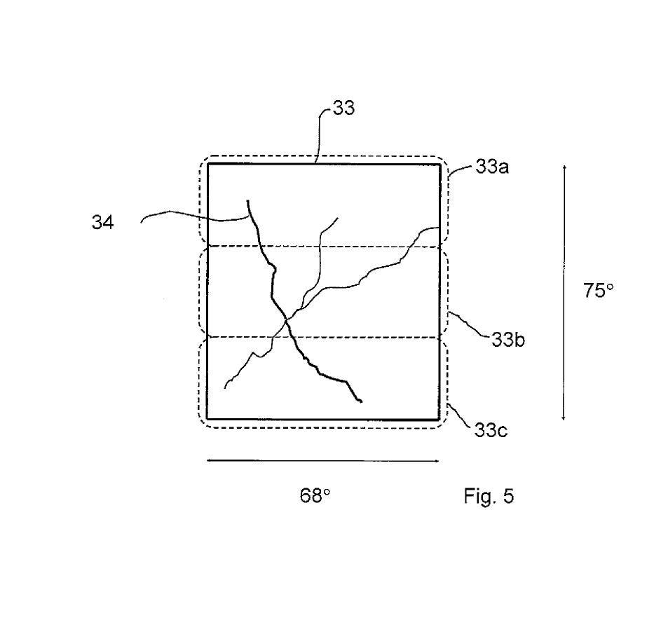

FIG. 5, a highly simplified representation of a photographic recording of an enlarged region of the retina produced with the fundus camera according to the invention;

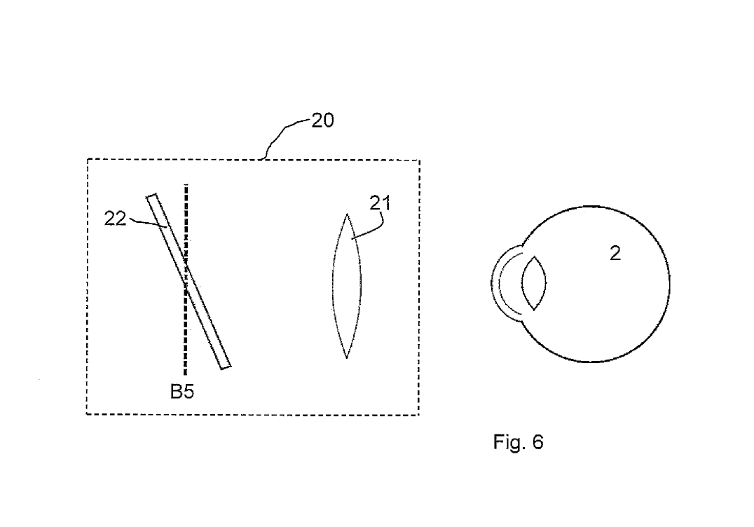

FIG. 6, one feasible embodiment of the patient guidance module according to the invention for use with a fundus camera making possible a variable focusing;

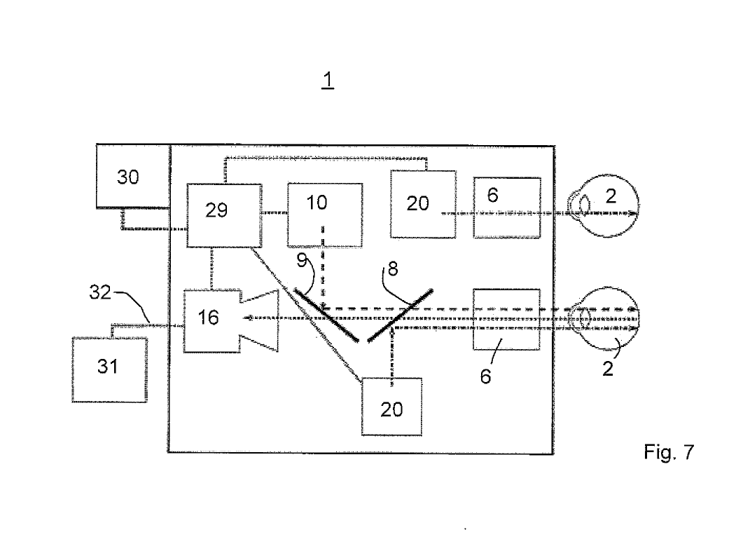

FIG. 7, an example of a fundus camera with binocular patient guidance according to the present invention, and

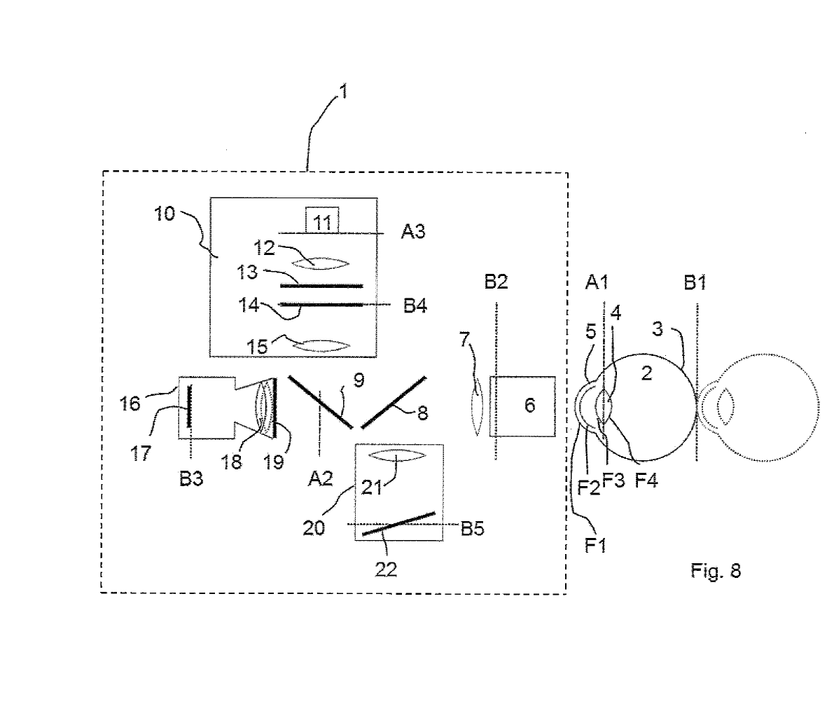

FIG. 8, a representation of another example of a fundus camera according to the invention.

Reference number 1 in FIG. 1 designates a fundus camera according to the invention in its entirety. The fundus camera 1 serves to examine the back of an eye 2 and to prepare photographic recordings thereof.

When the fundus of the eye 2 is lighted, photography-related image disturbances (artifacts) occur in the fundus image, which are caused by the back reflection of the lighting source. These artifacts are also called the “first to fourth Purkinje reflections”. The origins of these Purkinje reflections R1-R4 are designated as F1-F4 in FIG. 1. They occur at various places in the eye. The first Purkinje reflection R1 occurs at the front side of the cornea 5 and is by far the strongest. The second Purkinje reflection R2 occurs at the back side of the cornea 5. The third and fourth Purkinje reflections R3 and R4 occur due to focusing and scattering on the front side and back side, respectively, of the lens 4 of the eye 2.

The fundus camera 1 comprises a lighting module 10 with an approximately punctiform light source 11, preferably in the form of an LED module. The LED module comprises three emitters, with the colors infrared, green and ultraviolet. The use of the colors infrared, green and ultraviolet makes it possible to use a black and white camera, which has a higher light sensitivity than a color camera, so that the intensity of the lighting and thus the burden on the patient can be reduced. The turning of the individual LED emitters on and off is controlled by a microcontroller 29 and a computer 31 (see also FIG. 7).

The collimators 12 and 15 project the approximately punctiform light source 11 on the mirror 9. A polarization filter 13 together with another polarization filter 19 positioned in front of the digital camera 16 serves to moderate reflections in an eyepiece 6 and in the eye 2. Moreover, a slit diaphragm 14 is provided, by means of which a striplike lighting region is created and projected onto the fundus 3 (see region 2 in FIG. 2).

The device used to produce digital photographic recordings of the fundus 3 is a digital camera 16, preferably a digital black and white camera. The digital camera 16 has a resolution of two megapixels, for example. The digital camera 16 comprises a photosensitive electronic component 17 for detecting two-dimensional electronic images. Furthermore, the digital camera 16 comprises an objective 18. An autofocus unit can be integrated in the digital camera 16 to equalize spherical vision defects. The fundus camera 1 furthermore comprises the eyepiece 6, which is adapted to the design of the fundus camera 1 by a field lens 7.

A patient guidance module is designated as 20. This serves to produce a guided changing of the patient's viewing direction by means of a fixation mark generated by the patient guidance module 20. In this way, selected regions of the fundus can be imaged, i.e., detected. Thus, partial recordings can be merged to form a comprehensive overall representation of the fundus.

Reference number 22 designates a display, preferably a so-called OLED display, for generating the electronic fixation mark. The display projection optics 21 connected upstream from the display 22 projects the display 22 with the fixation mark generated on it onto the intermediate image plane B2 in the eyepiece 6.

The beam splitter 8 serves to integrate the patient guidance module 20 in the layout.

In the optical layout of the fundus camera 1 there are the image planes B1 to B5, which are projected one into another by lenses. The image plane B1 is situated on the fundus 3 of the patient. The patient “sees” this image. The image plane B2 constitutes an intermediate image inside the eyepiece 6, whose image section is limited in the horizontal direction by the field diaphragm, in the example of FIG. 2 to a circular image section corresponding to a viewing angle of auf 68°. The image plane B3 constitutes the image which is situated on the photosensitive electrical component 17 of the digital camera 16. This image of the image plane B3 is “seen” by the camera. The image plane B4 projects the homogeneous illuminated gap of the slit diaphragm 14. The image plane B5 is the image plane of the display 22, in which the fixation mark is generated.

If an object is situated in one of the image planes B1 to B5, it will be projected in all the other image planes. From image plane B1 to image plane B3, the fundus will be projected on the photosensitive electronic component 17 of the digital camera 16. From image plane B4 to image plane B1, the homogeneously lighted gap of the slit diaphragm 14 will be projected on the fundus 3 under uniform lighting. From image plane B5 to image plane B1, a fixation mark shown by the display 22 will be projected on the fundus 3 by the display projection optics 21 and the beam splitter 8.

FIG. 2 shows an example of the striplike division of the image area 23 in the image plane B1. In the middle of the image area 23 is situated the lighted strip 24 as the projection of the slit diaphragm 14 of the lighting module 10. At the top and bottom side of the lighted strip 24 there is an unlighted region 28a and 28b. The lighted strip 24 in the example shown thus corresponds to an image angle in the horizontal direction of around 68° and an image angle in the vertical direction of 30°, for example. Thanks to the striplike definition of the image field 23, the artifacts which are caused by the Purkinje reflections R3 and R4 can be moved into the unlighted region 28a and 28b. This prevents the Purkinje reflections R3 and R4 from affecting the lighted region of the fundus. The image area 23 is defined by the full circular region inside the field diaphragm of the eyepiece 6. The illuminated strip 24 can be stationary with regard to its principal direction in the fundus camera 1, for example; that is, it does not undergo any translatory movement. Advisedly, however, a changing of the slit width at the slit diaphragm 14 can be provided. As is seen from the photographic representation of FIG. 4, artifacts caused by Purkinje reflections, such as the third and/or fourth Purkinje reflection R3 and R4, are situated in the region 28a and/or 28b. The illuminated strip 24, on the contrary, is free of reflections of the approximately punctiform light source on the lens of the eye. The remaining reflections R5 and R6 have their origin in the eyepiece 6.

Another group of planes which are projected one into another in the layout are the aperture planes A1 to A3. The aperture plane A1 involves the pupil of the eye. In the aperture plane A2, a separation of the observation and lighting beam paths is done by the mirror 9. The aperture plane A3 is the plane of the light source 11 with the LED module. The configuration of the aperture planes A1 to A3 makes it possible first and foremost to fade out the artifacts of the first and second Purkinje reflection R1 and R2, that is, the Purkinje reflections R1 and R2 are extinguished even before they are projected onto the image sensor 17 or at least the intensity of the artifacts caused by the Purkinje reflections is reduced in part.

The mirror 9 performs a separation of the observation and lighting beam paths, as is shown in FIG. 3. Reference number 25 in FIG. 3 designates the pupil, i.e., the natural opening through which light can enter into the eye 2. The mirror 9 divides the area of the pupil 25 into a circular segment observation region 26 and a circular segment lighting region 27. In the lighting region 27 lies the image of the approximately punctiform light source 11. In FIG. 3, the two regions 26 and 27 are shown as semicircular, for example. These two regions could just as easily be in the shape of a circular section. The aperture division of the pupil 25 per FIG. 3 is preferably constant. The apparatus layout in this respect is stationary. Only the lens of the objective 18 of the digital camera 16 can be moved for focusing. This results in an especially simple, low-cost layout of the fundus camera 1.

By means of the aperture division per FIG. 3, the first and the second Purkinje reflection R1 and R2 are eliminated or at least the intensity of the artifacts caused by the Purkinje reflections is diminished. The aperture plane A1 lies on the surface of the lens 4 in the region of the third Purkinje reflection R3, so that a sharp projection of the Purkinje reflections R3 and/or R4 and thus a limiting of the image area affected by them results, as is evident from FIG. 4. By orienting the layout such that the third and/or fourth Purkinje reflections R3 and R4 are projected into the unused regions 28a and 28b per FIG. 2, the aforementioned reflections can be almost entirely eliminated from the picture. Since an approximately punctiform light source 11 is being used and the Purkinje reflections R3 and/or R4 caused by the use of the approximately punctiform light source are relatively sharply projected into the regions 28a and 28b, furthermore the relative position of the fundus camera to the eye can also be inferred by evaluating the position of the Purkinje reflection R3 and/or R4 in the image. This gives the possibility of a centering of the fundus camera 1 on the eye 2, especially an automatic centering.

The fundus camera 1 according to the invention makes it possible to substantially enlarge the area region of the fundus 3 being examined by guiding the eye 2 in movement in the aforementioned stationary, i.e., static layout of the lighting beam path and the detection beam path, so that the region of the fundus 3 of the patient is shifted under the lighted strip 24 of the image area 23 and the area there in the image area 23 is changed. This makes it possible to record vertical regions of the fundus 3 of far more than 30°. In this way, with simple means, the possibility is created of examining extensive surface regions of the fundus 3 without the use of pupil-widening agents (mydriatica), so that an increased patient comfort is achieved and no costly layouts involving movement mechanics have to be used.

The patient guidance module 20 provided for such patient guidance (see FIG. 1) makes it possible to generate a fixation mark (light mask) on the fundus 3 in the image plane B1. By changing the fixation mark, the patient is caused to change the position of the eye as compared to the digital camera 16 according to the position change of the fixation mark, for example, by following the fixation mark vertically upward or downward and thereby changing the region of the fundus 3 being studied under the lighted strip 24. In combination with an actuating of the patient guidance, the viewing direction of the patient can be controlled so that different regions of the retina are projected and these are merged (stitching) by combination to form a larger overall image. In this way, a region of 68° or more in the vertical direction can also be achieved. FIG. 5 shows in highly simplified schematic representation a corresponding recording 33 produced by means of patient guidance in an enlarged angle region of the fundus 3. The recording 33 is composed of three individual recordings 33a-33c, which have been merged to form the recording 33. Blood vessels visible in the recording are designated by 34.

FIG. 6 shows the principle of the patient guidance module 20 to enable a focusing, i.e., an adaptation of the patient's accommodation without moving parts. This is accomplished by a slanted position of the display 22 relative to the image plane B5. The variation of the fixation direction is achieved by a vertical movement, i.e., a movement perpendicular to the plane of the drawing in FIG. 6 of the light point or pixel serving as the fixation mark. The variation of the accommodation and the correction of spherical vision defects on the other hand is accomplished by a horizontal movement, i.e., by a movement parallel to the plane of the drawing of FIG. 6. Thanks to the slanted position of the display 22 with respect to the image plane B5, the distance to the display projection optics 21 changes during horizontal movement of the image point (shifting of the image point parallel to the plane of the drawing of FIG. 6). A patient guidance module 20 shown accordingly in FIG. 6 is preferably provided for each eye, in order to vary the accommodation and correct for any vision defects.

The configuration shown in FIG. 7 for a fundus camera 1 with binocular patient guidance comprises a patient guidance module 20 of the described kind for each eye 2 and a microcontroller 29, which controls the movement of the fixation marks in the patient guidance module 20 associated with the particular eye 2. A detection beam path between the eye 2 and the digital camera 16 is provided only for one eye 2. The digital camera is connected by a suitable data line 32, for example by an Ethernet interface, to a computer 31. Thanks to a positioning unit 30, the fundus camera 1 can be optimally oriented to the eyes. Preferably, the positioning unit is motorized and can be controlled automatically or manually by the microcontroller 29 and the computer 31. As the basis for an automatic control, one can use the position of the artifacts of the Purkinje reflections R3 and/or and R4 in the image field 23.

The overall layout of the fundus camera in FIG. 7 can be swiveled about an axis (not shown) running between the two eyes by 180°, so that instead on the one eye the other eye can also be examined.

In one-eyed patients, only the imaging part of the layout will be used.

The microcontroller 29 furthermore receives control commands from the computer 31 in order to turn the LEDs of the light source 11 on and off in the proper sequence. At the same time, the digital camera 16 is triggered by the computer 31 so that lighting and exposure occur in synchronization. The digital camera 16 has inputs and outputs to enable a communication and relaying of control commands from the computer 31 to the microcontroller 29 as well as synchronize the lighting and the exposure. Alternatively, the computer can also be connected directly to the microcontroller 29. An autofocus unit can be integrated in the digital camera 16 in order to correct for spherical vision defects.

With the help of software, gray-scale images sequentially obtained are combined on the computer into a single color image. For this, it is necessary to detect any offset present in the images (registration), compensate appropriately for it, and then combine the images into a single color picture. The computer is outfitted with a user interface, which enables attendance and control of the fundus camera as well as display of the fundus recordings.

Photographic observation and/or documentation in the sense of this invention includes both photographic recordings and film or video recordings.

In the simple operating mode of the fundus cameras, see FIG. 1, this only projects the fundus 3 with the retina. In this operating mode of the fundus camera, the image plane B1, as shown in FIG. 1, lies on the fundus 3 or the retina of the eye 2 and the aperture plane A1 lies in or near or within the lens of the eye 4.

In an expanded operating mode, the distance of the fundus camera 1 from the eye 2 can be increased and/or the focus setting of the objective 18 can be varied, as is shown in simplified manner in FIG. 8 (see the dotted-line positions of the eye 2 and the objective 18). Preferably, both processes can be attuned to each other.

Alternatively or in addition to the aforesaid measures, the orientation of the fundus camera 1 to the eye 2 can also be changed. By orientation is meant (not being shown in FIG. 8) the angle position of the optical axis of the fundus camera 1 to the optical axis of the eye 2, the angle position of the optical axis of the fundus camera 1 to the optical axis of the eye 2 and/or a rotation of the fundus camera 1 about its optical axis. By optical axis of the fundus camera 1 is meant in particular the optical axis of the eyepiece 6.

In this way, the image plane B1 can be shifted into any given regions in the eye to the front as far as the region FI. In this way, when B1 comes to lie in the corresponding region, it is possible to project, for example, regions of the vitreous humor, which is the region between F4 and the fundus 3 in FIG. 1 and FIG. 8, the lens of the eye 4, the anterior chamber, the iris, which is the region between F2 and F3 in FIG. 1, and/or the cornea, which is the region between F1 and F2 in FIG. 1. This makes it possible to further enhance the degree of examination options for the eye 2.

The aperture plane A1 in this expanded operating mode is likewise shifted forward in the eye 2, up to a position outside the eye 2, i.e., it is shifted starting from a position in or near the lens 4 in the direction of the cornea 5 and finally into a region between eye 2 and fundus camera 1.

### LIST OF REFERENCE SYMBOLS

1 Fundus camera
2 Eye
3 Fundus (back of the eye)
4 Lens
5 Cornea
6 Eyepiece
7 Field lens
8 Beam splitter
9 Mirror
10 Lighting module
11 Light source
12 Collimator
13 Polarization filter
14 Slit diaphragm
15 Collimator
16 Digital camera
17 Image sensor
18 Objective
19 Polarization filter
20 Patient guidance module
21 Display-projection optics
22 Display
23 Image area
24 Illuminated strips
25 Pupil
26 Observation region
27 Lighting region
28 Unlit region
29 Microcontroller
30 Positioning unit
31 Computer
32 Data line
33 Recording
34 Blood vessel
A1-A3 Aperture planes
B1-B5 Image planes
F1-F4 Origins of the first to fourth Purkinje reflections
R1-R4 First to fourth Purkinje reflections
R5-R6 Reflections of the eyepiece

1-34. (canceled)

35. A method for photographic observation and/or documentation of the fundus of an eye, which comprises the following steps:
producing a lighting beam path by means of a lighting module for lighting at least one partial area of the fundus by using an at least approximately punctiform or at least areally limited lighting source,
producing an image plane in the lighting beam path for the creation of photographic recordings,
separating the pupil cross sectional area into a detectable observation region and an undetectable lighting region,
producing an image area that contains the image of the fundus on the image plane,
dividing the image area in strips in the lighting beam path into a lighted and unlighted regions,
projecting at least one Purkinje reflection onto the image area, wherein the lighting beam path is stationary relative to the overall layout of the fundus camera and the at least one Purkinje reflection is located essentially in the unlighted regions of the image area.

36. The method according to claim 35, wherein the detectable observation region and the undetectable lighting region of the pupil cross section area have a semicircular or circular section shape.

37. The method according to claim 35, wherein the aperture plane A1 of the pupil is displaced backward into the eye, preferably into a plane at the front side of the lens of the eye or at least into a plane in the region of the lens of the eye.

38. The method according to claim 35, wherein the third and/or fourth Purkinje reflections are sharply projected at least essentially onto the image plane.

39. The method according to claim 35, wherein the lighted region of the image area corresponds to an image angle in the transverse direction to the strip of at most 5°-30°, preferably 20°.

40. The method according to claim 35, wherein the lighted region of the image area corresponds to an image angle in the longitudinal direction to the strip of 30°-80°, preferably 68.

41. The method according to claim 35, wherein the detection beam path is stationary relative to the overall construction of the fundus camera.

42. The method according to claim 35, wherein the area of the fundus being examined in the lighting beam path and detection beam path is varied by changing the viewing direction of the patient's eye relative to the orientation of the lighting beam path and/or detection beam path.

43. The method according to claim 42, wherein the different regions of the fundus due to the changing of the viewing direction of the patient's eye are detected separately and combined into a total image.

44. The method according to claim 35, wherein a patient guidance module is provided, by means of which the viewing direction of the patient is guided by means of a fixation mark in the eye being examined or in the other eye, so that by changing the viewing direction regions of the fundus lying outside the detection are shifted into the region of the detection.

45. The method according to claim 35, wherein a monocular or binocular guidance of the patient's eye is provided by projecting a fixation mark onto the fundus of one eye or both eyes.

46. The method according to claim 45, wherein a pair of stereo images is used for the fixation marks of both eyes in the case of binocular guidance of the eye.

47. The method according to claim 45, wherein the fixation mark is sharply projected onto the retina in the case of spherical vision defect of the eye.

48. The method according to claim 44, wherein a deliberate stimulation of the accommodation state is done, preferably a deaccommodation.

49. The method according to claim 35, wherein the positions and/or forms of the third and/or fourth Purkinje reflection are used in the fundus image for determining the spatial position of the detection optics relative to the lens of the eye.

50. The method according to claim 49, wherein the relative spatial position which is determined is used for positioning the fundus camera relative to the eye.

51. A fundus camera for photographic observation, documentation and/or diagnosis of the retina of an eye, comprising
a lighting module with preferably approximately punctiform or at least areally limited lighting source to produce a lighting beam path,
a slit diaphragm for confining the lighting beam path to a lighted strip of the fundus next to unlighted regions,
a digital camera,
a detection beam path between the digital camera and the fundus, wherein
a divider is provided for dividing the detection beam path into a detectable observation region and an undetectable lighting region, wherein
at least one Purkinje reflection is projected each time in the unlighted region and
the lighting beam path is stationary relative to the overall construction of the fundus camera.

52. The fundus camera according to claim 51, wherein the detection beam path is stationary relative to the overall construction of the fundus camera.

53. The fundus camera according to claim 51, wherein the detectable observation region and the undetectable lighting region have a semicircular or circular section shape.

54. The fundus camera according to claim 51, wherein a mirror is provided for the division of the detection beam path and the first and second Purkinje reflection lie in the undetectable lighting region.
